(19) United States
(12) Patent Application Publication
(10) Pub. No.: US 2008/0139896 A1
(43) Pub. Date: Jun. 12, 2008

(54) System and Method for Graphical Annotation of Anatomical Images Using a Touch Screen Display

(75) Inventor: John Baumgart, Hoffman Estates, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 11/610,658

(22) Filed: Dec. 14, 2006

Related U.S. Application Data
(60) Provisional application No. 60/829,370, filed on Oct. 13, 2006.

(51) Int. Cl. A61B 5/00

(52) U.S. Cl. 600/300

(57) ABSTRACT

A system for graphically annotating an anatomical image includes a main image display; a touch-screen display; a processing unit; and a network configured to interface the processing unit with the main image display and with the touch-screen display. An image displayed on the main display is concurrently displayed on the touch-screen display. The touch-screen display enables dragging and dropping at least one graphical annotation tool displayed on the main display from the main display to the anatomical image concurrently displayed on the touch-screen display by touching the graphical annotation tool displayed on the touch-screen display. Touching of the annotation tool to drag and drop can be effected by a user touching the touch-screen display, by a stylus, by a light pen, by a mouse, by a track ball, and by a joystick control. A corresponding method is also disclosed, and also a corresponding touch-screen display.

### CROSS-REFERENCE TO RELATED APPLICATIONS

This application claims priority under 35 U.S.C. §119 to U.S. Provisional Patent Application Ser. No. 60/829,370 filed on Oct. 13, 2006 entitled “Graphical Annotation of X-ray Images Using a Touch-Screen”, the entire contents of which are incorporated by reference herein. This application cross-references concurrently filed U.S. patent application Ser. No. (Attorney Docket Number 2006P22073US01) by John Baumgart, entitled “System and Method for Selection of Anatomical Images for Display Using a Touch-Screen Display”, the entire contents of which is incorporated by reference herein and concurrently filed U.S. patent application Ser. No. (Attorney Docket Number 2006P22072US01) by John Baumgart, entitled “System and Method for Selection of Points of Interest During Quantitative Analysis Using a Touch-Screen Display”, the entire contents of which is incorporated by reference herein.

### BACKGROUND

1. Technical Field

The present disclosure relates to medical imaging systems.

2. Discussion of Related Art

In current analysis of anatomical images, e.g., X-ray images, in an examination room, a bedside touch-screen display is used to select, place and size graphical objects on an image that is displayed on a main panel display that is positioned on an opposite side of the examination bed in the examination room. The touch-screen display includes only a joystick control or mouse that the user must use at the bedside position. Using the joystick to position the pointer over an appropriate icon on the display to annotate an image is slow and awkward.

Therefore, a need exists for positioning and displaying ideas on a display locally and remotely.

### SUMMARY

The present disclosure relates to a system for graphically annotating an anatomical image. The system includes a main image display; a touch-screen display; a processing unit; and a network configured to interface the main image display with the processing unit and configured to interface the touch-screen display with the processing unit. The system is configured such that an image displayed on the main display is concurrently displayed on the touch-screen display via the processing unit interfacing the main image display with the touch-screen display through the network. The system is also configured such that the touch-screen display enables dragging and dropping at least one graphical annotation tool displayed on the main display from the main display to the anatomical image concurrently displayed on the touch-screen display by touching the at least one graphical annotation tool displayed on the touch-screen display.

The touching of the at least one graphical annotation on the touch-screen display may be effected by one of a stylus; a light pen; a mouse; a display screen thumbnail; a track ball; a joystick control; and the touch-screen display being touched by a user. The touch-screen display may be configured to enable at least one of positioning and varying of size of the at least one graphical annotation. The system may be configured such that following graphical annotation of the anatomical image on the touch-screen display, the main display displays quantitative results of the at least one graphical annotation on the anatomical image. In addition, the system may be configured such that following graphical annotation of the anatomical image on the touch-screen display, the touch-screen display displays a display mode displayed prior to the display of the at least one graphical annotation tool. The system may further include a patient bed, wherein the touch-screen display is positioned in proximity to the patient bed.

The present disclosure relates also to a method for graphically annotating an anatomical image. The method includes the steps of: providing a main image display and a touch-screen display; displaying concurrently an image displayed on the main display on the touch-screen display; and dragging and dropping at least one graphical annotation tool displayed on the main display from the main display to the anatomical image concurrently displayed on the touch-screen display by touching the at least one graphical annotation tool displayed on the touch-screen display.

The method may be implemented wherein the dragging and dropping of the at least one graphical annotation tool on the touch-screen display is effected by touching the touch-screen display via one of a stylus; a light pen; a mouse; a display screen thumbnail; a track ball; a joystick control; and touching by a user. The method may include the steps of at least one of positioning and varying of size of the at least one graphical annotation on the touch-screen display. In addition, following at least the dragging and dropping of the at least one graphical annotation tool to the anatomical image on the touch-screen display, the method may include the step of displaying on the main display quantitative results of the at least one graphical annotation on the anatomical image or displaying on the touch-screen display a display mode displayed prior to the display of the at least one graphical annotation tool.

The present disclosure also relates to a touch-screen display for graphically annotating an anatomical image. The touch-screen display is configured to interface with a main image display; a processing unit; and a network configured to interface the main image display with the processing unit and configured to interface the touch-screen display with the processing unit. The touch-screen display is configured such that an image displayed on the main display is concurrently displayed on the touch-screen display via the processing unit interfacing the main image display with the touch-screen display through the network, and the touch-screen display is configured such that the touch-screen display enables dragging and dropping at least one graphical annotation tool displayed on the main display from the main display to the anatomical image concurrently displayed on the touch-screen display by touching the at least one graphical annotation tool displayed on the touch-screen display.

The touching of the at least one graphical annotation on the touch-screen display may be effected by one of a stylus; a light pen; a mouse; a display screen thumbnail; a track ball; a joystick control; and touching by a user. The touch-screen display may be configured to enable at least one of positioning and varying of size of the at least one graphical annotation. The touch-screen display may also be configured such that following graphical annotation of the anatomical image on the touch-screen display, the main display displays quantitative results of the at least one graphical annotation on the anatomical image. In addition, the touch-screen display may be configured such that following graphical annotation of the anatomical image on the touch-screen display, the touch-screen display displays a display mode displayed prior to the display of the at least one graphical annotation tool. The touch-screen display may be positioned in proximity to a patient bed.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure:

FIG. 1 is an overview of an exemplary angiographic X-ray system in a patient examination room illustrating a system user, a patient lying on a bed, main X-ray displays and a touch-screen display at the bedside;

FIG. 2 is a perspective view of the main panel displays;

FIG. 3 is a perspective view of a touch-screen display according to the present disclosure before or after a graphical annotation process;

FIG. 4A illustrates a first exemplary graphical subtask card of graphical annotation tools that are displayed on the main panel display that are now available on the touch-screen panel according to the present disclosure;

FIG. 4B illustrates a second exemplary graphical subtask card of graphical annotation tools that are displayed on the main panel display that are now available on the touch-screen panel according to the present disclosure; and

FIG. 5 is a perspective view of an exemplary image and graphical subtask card on the touch-screen display of FIG. 3 according to the present disclosure.

### DETAILED DESCRIPTION OF EXEMPLARY EMBODIMENTS

Exemplary embodiments of the present disclosure will now be described in detail with reference to the figures, in which like reference numerals identify corresponding elements throughout the several views.

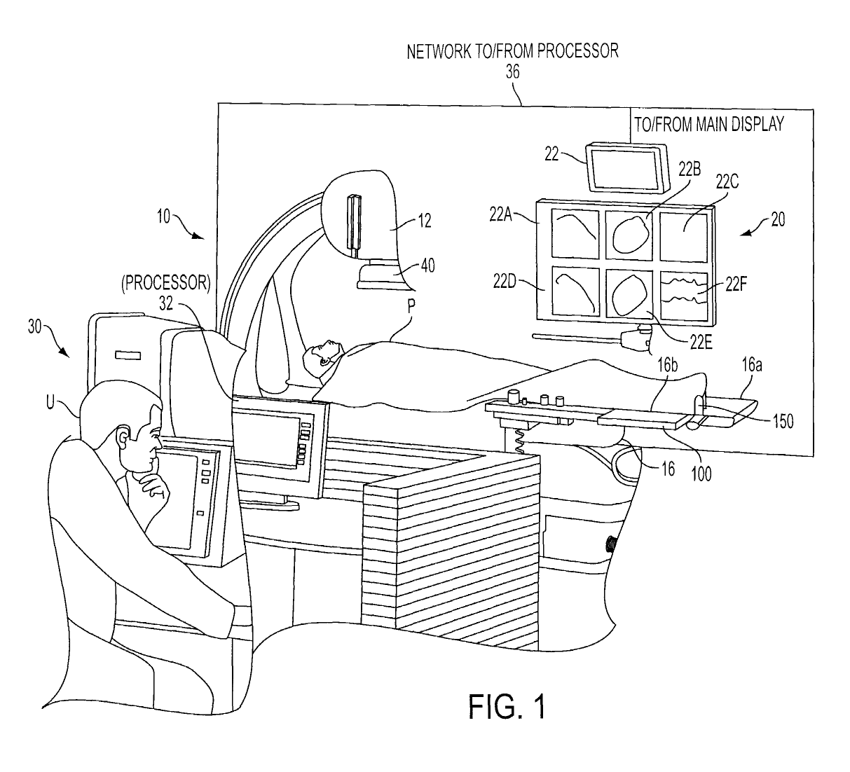

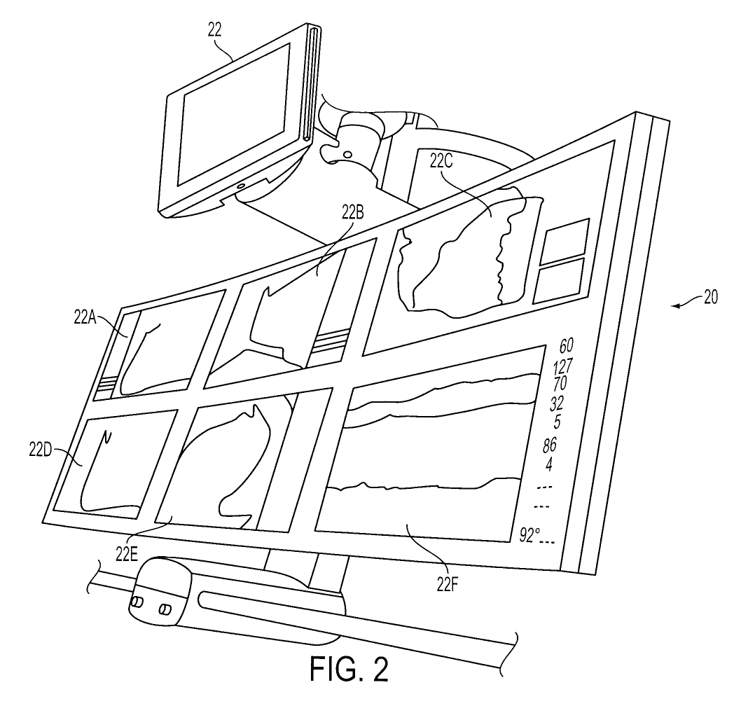

Referring to FIGS. 1-5, there is illustrated an exemplary angiographic x-ray system 10 as disposed in a patient examination room. A patient P is positioned on an examination bed 16. The x-ray or radiographic system 10 includes an image detector 40 supported by a support structure 12 and positioned over the examination bed 16. The image detector 40 is positioned over the patient P and over the examination bed 16 to detect the x-rays emitted from an x-ray source (not shown) under the bed 16 that enable recording the anatomical images. The radiographic system 10 includes a bank of main panel displays 20, e.g., overhead panel 22 and individual panel displays, e.g. panel displays 22A, 22B, 22C, 22C, 22D, 22E and 22F (see FIG. 2). The patient P and the main panel displays 20 are within view of a user U, e.g., a physician, seated at a control console 30. The main panel displays 20 are disposed on a distal side 16a of the examination bed 16 with respect to the user U.

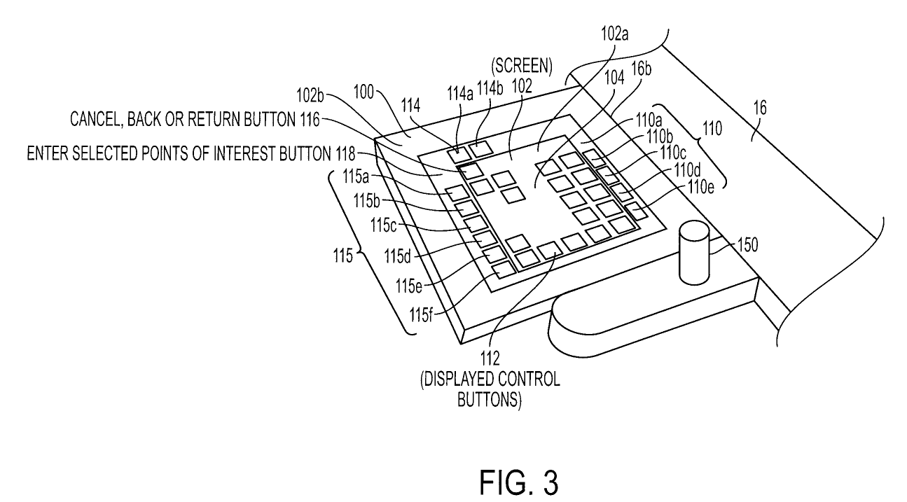

As best illustrated in FIG. 3, the bed 16 includes a touch-screen display 100 according to the present disclosure with a joystick control 150, each disposed on a proximal side 16b of the examination bed 16. The touch-screen display 100 includes a screen 102 that is sensitive to touch. The screen 102 may be subdivided into a main portion 102a and a border portion 102b around the main portion 102a. The user U may be standing at the proximal side 16b of the bed 16, where the touch-screen display 100 is located, and from which location the main panel or image displays 20 are also within view of the user U.

Referring to FIG. 1, the radiographic system 10 further includes a processing unit 32 that may be located at the control console 30 and a network 36 that is configured to interface the main image display 20 with the processing unit 32 and is also configured to interface the touch-screen display 100 with the processing unit 32.

In one embodiment, an upper edge of the border portion 102b of the screen 102 includes a strip 110 of touch buttons or tabs, e.g., touch buttons 110a, 110b, 110c, 110d, and 110e, that is disposed proximate to the proximal edge 16b of the bed 16. The border portion 102b further includes a strip 114 of touch buttons or tabs, e.g., touch buttons 114a and 114b disposed on the left side of the border portion 102b and a strip 115 of touch buttons 115a, 115b, 115c, 115d, 115e and 115f disposed on the bottom side of the border portion 102b.

When one of the touch buttons, e.g., touch button 110b, is pressed on the screen 102, a particular series 112 of control buttons is displayed on the main portion 102a of the screen 102, as shown. If another touch button, e.g., touch button 110d is touched, a different series of control buttons (not shown) is displayed on the main portion 102a of the screen 102. However, the configuration, function, and position of touch buttons 114 on the border portion 102b does not change by touching the buttons 110a through 110e. In addition to the strips 110 and 114 of touch buttons, the screen 102 of the touch-screen display 100 further includes a “back”, “cancel”, or “return” button 116 that may be disposed in the border region 102b. The function of the “back”, “cancel”, or “return” button 116 is discussed in more detail below. Similarly, the screen 102 further includes an “Enter Graphical Annotation Tool” button 118. The function of the button 118 is also discussed in more detail below.

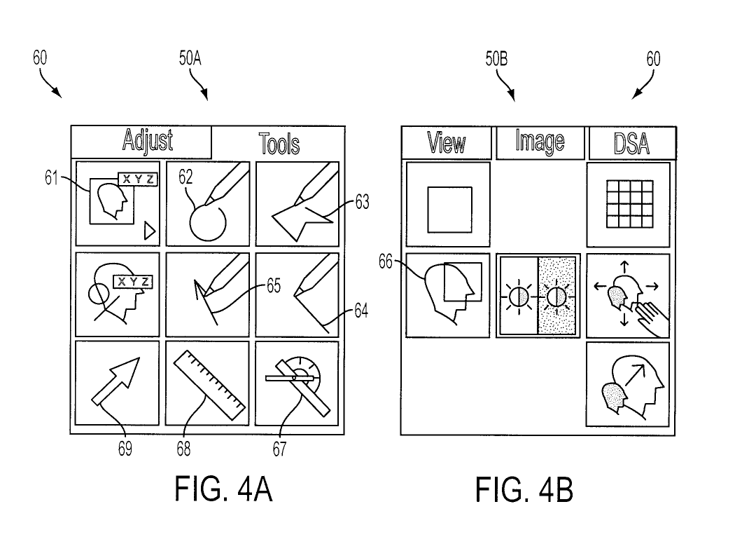

FIGS. 4A and 4B are views of exemplary graphical subtask cards 50A and 50B illustrating graphical annotation tools or graphical element tools 60 that are displayed on the main panel display 20 that are now available for dragging and dropping to the touch-screen panel 100 according to the present disclosure. The graphical annotation tool or graphical element tool 60 may include annotation markings 61, circles 62, polygons 63, lines 64, arrows 65, electronic shutter 66, angles 67, distances 68 and pointers 69.

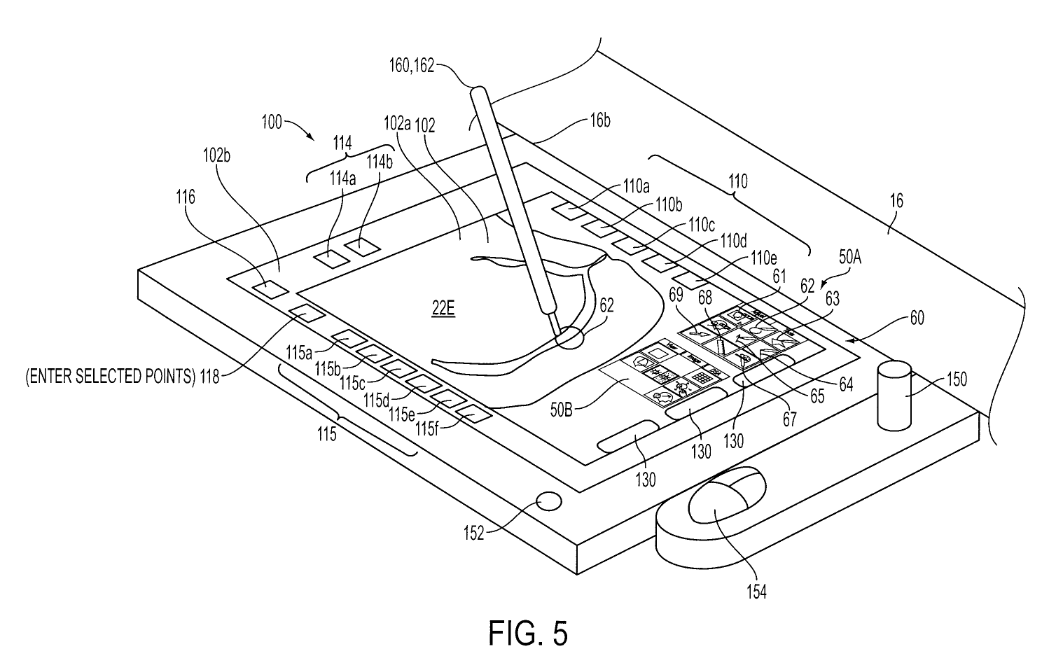

More particularly, referring to FIG. 5, the system 10 is configured according to the present disclosure such that an anatomical image, e.g., images 22, or 22A to 22F (see FIGS. 1 and 2), displayed on the main display 20 is concurrently displayed on the touch-screen display 100 through the network 36. The touch-screen display or console 100 is positioned in proximity to the bed 16, e.g., on the side 16b thereof, as illustrated in FIG. 5, at which position the user U may stand. In addition, the system 10 is configured such that at least one of the graphical subtask cards 50A or 50B is now capable of being displayed on the screen 102 of the touch-screen display 100. Therefore, the system 10 is configured such that the touch-screen display 100 enables dragging and dropping at least one graphical annotation tool or graphical element tool, e.g., circle 62 of tools 60 illustrated on graphical subtask card 50A in FIG. 5, that is displayed on the main display 20 to the anatomical image, e.g., images 22C, concurrently displayed on the touch-screen display 100 by touching the at least one graphical annotation tool or graphical element tool, e.g., circle 62, displayed on the screen 102 of the touch-screen display 100.

The touch-screen display 100 may include, in addition to the joystick 150, a track ball 152 and/or a mouse 154. The mouse 154 may be wired to the processor 32 or may be operatively coupled to the processor 32 wirelessly or contactlessly, e.g., via optical, electromagnetic, or acoustic waves. The mouse 154 may also be configured as a “built-in” touch panel and scroll design available for portable computers. The mouse 154 may be configured to interface concurrently with the main display 20 and the touch-screen display 100 via the processing unit 32 and the network 36 that is configured to interface the main image display 20 with the processing unit 32 and that is configured to interface the touch-screen display 100 with the processing unit 32. The embodiments are not limited in this context.

The touch-screen display 100 may also be operatively coupled to the processor 32 via a stylus 160 or light pen 162 (concurrently illustrated). The screen 102 may also display on at least one edge one or more thumbnails 130 that may be pressed for selection of the image, e.g., image 22E that is currently displayed on the main display 20 to be simultaneously displayed on the screen 102. The embodiments are not limited in this context.

The user U can use the stylus 160 or the light pen 162 to drag and drop directly on the touch-screen 102 a graphical annotation tool or graphical element tool 60 from at least one of the subtask cards 50A or 50B to the anatomical image, e.g., image 22E, displayed on the screen 102 of the touch-screen display, thereby effecting touching of the touch-screen 102. The user U can also touch the screen 102 using a part of the user's body, e.g., one or more fingers, to perform the dragging and dropping of the graphical annotation tool or element 60. In addition, the user U can use the track ball 152 or the mouse 154 also to perform the dragging and dropping on the touch-screen 102 of the tool or element 60 to the anatomical image, e.g., image 22E, thereby effecting the touching of the touch-screen 102. As defined herein, touching of the touch screen 102 to perform the dragging and dropping of the graphical annotation tool or element 60 may also be effected by the joystick control 150. The system 10 is also configured such that the touch-screen display 102 enables at least positioning and/or sizing of the graphical annotation or element tool 60, as desired. The positioning refers to changing the position of the annotation or element tool 60 to a desired position while the sizing refers to changing the size of the annotation or element tool 60 to a desired size.

Once the user U has definitively selected the graphical annotation or element tool 60 desired and positioned or sized the tool 60 as desired, the user U can then press the “Enter Graphical Annotation Tool” button 118 on the touch-screen display 100 to enable the software to perform quantitative analysis associated with the selected graphical annotation tool or element 60.

Following selecting the at least one graphical annotation or element tool 60, the system 10 is configured such that the quantitative results associated with the selected graphical annotation or element tool 60, e.g., circle 62, are displayed on the main display 20. Also following the desired selecting and positioning or sizing the at least one annotation or element tool 60, a display mode displayed prior to the display of the at least one graphical subtask card 50A and 50B and of the anatomical image 22 or 22A to 22F, e.g., display mode 104 illustrated in FIG. 3, is displayed on the touch-screen display 100.

Although the anatomical image described herein is disclosed with respect to exemplary angiographic x-ray system 10, the embodiments of the present disclosure may be applied to other anatomical images and imaging systems such as, but not limited to, computer assisted tomography (CAT), magnetic resonance imaging (MRI), positron emission tomography (PET) and acoustic or sonogram images.

Referring again to FIGS. 1-5, the present disclosure relates also to the touch-screen display 100 for graphically annotating an anatomical image, e.g., the anatomical image 22E that is now displayed concurrently or simultaneously on the main image display 20. The touch-screen display 100 is positioned in proximity to the bed 16, e.g., on side 16b thereof, at which position the user U may stand. The touch-screen display 100 is configured to interface with the main image display 20, the processing unit 32, and the network 36 that is configured to interface the main image display 20 with the processing unit 32 and configured to interface the touch-screen display 100 with the processing unit 32. The touch-screen display 100 is configured such that an image, e.g., image 22E, displayed on the main image display 20 is concurrently or simultaneously displayed on the touch-screen display 100 via the processing unit 32 interfacing the main image display 20 with the touch-screen display 100 through the network 36. The touch-screen display 100 is configured such that the touch-screen display 100 enables dragging and dropping at least one graphical annotation or element tool 60 displayed on the main display 20 from the main display 20 to the anatomical image, e.g., image 22E, concurrently displayed on the touch-screen display 100 by touching the at least one graphical annotation or element tool 60 displayed on the screen 102 of the touch-screen display 100.

As described above, the touching of the at least graphical annotation or element tool 60 on the touch-screen display 100 to perform the dragging and dropping of the graphical annotation tool or element 60 on the touch-screen display 100 may be effected by the stylus 160 or light pen 162, or by the mouse 154, a display screen thumbnail 130, the track ball 152, or the joystick control 150. The user U can also touch the touch screen 102 using a part of the user's body, e.g., one or more fingers, to perform the dragging and dropping of the graphical annotation tool or element 60. The touch-screen display 100 may be configured such that following desired selection and positioning or sizing of the at least one graphical annotation tool 60, the main display 20 displays quantitative results associated with the annotation or element tool 60 on the at least one image, e.g., image 22E. Also, the touch-screen display 100 may be configured such that following selection, positioning or sizing of the at least one tool 60, the touch-screen display 100 displays a display mode displayed prior to the display of the image, e.g., image 22E, and tool 60, e.g., display mode 104.

In addition, the present disclosure relates also to a method for graphically annotating an anatomical image, e.g., image 22E (see FIGS. 2 and 5). The method includes the steps of providing the main image display 20 and the touch-screen display 100, displaying concurrently the image 22E displayed on the main display 20 on the touch-screen display 100, and dragging and dropping at least one graphical annotation or element tool 60, displayed on the main display, from the main display to the anatomical image concurrently displayed on the touch-screen display 100 by touching the at least one graphical annotation or element tool 60 on the touch-screen display 100.

The method may be implemented such that the touching of the at least one tool 60 on the touch-screen display 100 may be effected by the stylus 160 or light pen 162, or by the mouse 154, a display screen thumbnail 130, the track ball 152, or the joystick control 150. The user U can also touch the touch screen 102 using a part of the user's body, e.g., one or more fingers, to drag and drop the at least one tool 60 on the touch screen 102. The method may include the step of at least one of positioning and sizing the graphical annotation or element tool 60. The method may be implemented such that following at least the dragging and dropping of the at least one graphical annotation or element tool 60 to the anatomical image, e.g., image 22E, on the touch-screen display 100, the method includes the step of displaying on the main display 20 quantitative results associated with the at least one graphical annotation tool 60 on the at least one image 22E on the main display 20. The method may also be implemented such that following at least the dragging and dropping of the at least one graphical annotation tool to the anatomical image, e.g., image 22E, on the touch-screen display 100, the method includes the step of displaying on the touch-screen display 100 a display mode that was displayed prior to the display of the anatomical points of interest, e.g., display mode 104.

It will be understood that various modifications may be made to the embodiments disclosed herein. For example, although the above embodiments are described with reference to one particular configuration of the system, method and touch-screen display, the embodiments of the present disclosure may find application in conjunction with a system, method and touch-screen display having many different configurations. Accordingly, it is contemplated that the disclosure is not limited to such an application and may be applied to various embodiments.

What is claimed is:

1. A system for graphically annotating an anatomical image, the system comprising:
a main image display;
a touch-screen display;
a processing unit; and
a network configured to interface the main image display with the processing unit and configured to interface the touch-screen display with the processing unit,
wherein the system is configured such that an image displayed on the main display is concurrently displayed on the touch-screen display via the processing unit interfacing the main image display with the touch-screen display through the network, and
wherein the system is configured such that the touch-screen display enables dragging and dropping at least one graphical annotation tool displayed on the main display from the main display to the anatomical image concurrently displayed on the touch-screen display by touching the at least one graphical annotation tool displayed on the touch-screen display.

2. The system according to claim 1, wherein the touching of the at least one graphical annotation on the touch-screen display is effected by one of (a) a stylus; (b) a light pen; (c) a mouse; (d) a display screen thumbnail; (e) a track ball; (f) a joystick control; and (g) the touch-screen display being touched by a user.

3. The system according to claim 1, wherein the touch-screen display is configured to enable at least one of positioning and varying of size of the at least one graphical annotation.

4. The system according to claim 1, wherein
the system is configured such that following graphical annotation of the anatomical image on the touch-screen display, the main display displays quantitative results of the at least one graphical annotation on the anatomical image.

5. The system according to claim 1, wherein
the system is configured such that following graphical annotation of the anatomical image on the touch-screen display, the touch-screen display displays a display mode displayed prior to the display of the at least one graphical annotation tool.

6. The system according to claim 1, further comprising a patient bed, wherein the touch-screen display is positioned in proximity to the patient bed.

7. A method for graphically annotating an anatomical image, the method comprising the steps of:
providing:
a main image display; and
a touch-screen display;
displaying concurrently an image displayed on the main display on the touch-screen display; and
dragging and dropping at least one graphical annotation tool displayed on the main display from the main display to the anatomical image concurrently displayed on the touch-screen display by touching the at least one graphical annotation tool displayed on the touch-screen display.

8. The method according to claim 7, wherein the dragging and dropping of the at least one graphical annotation tool on the touch-screen display is effected by touching the touch-screen display via one of (a) a stylus; (b) a light pen; (c) a mouse; (d) a display screen thumbnail; (e) a track ball; (f) a joystick control; and (g) touching by a user.

9. The method according to claim 7, further comprising the steps of at least one of positioning and varying of size of the at least one graphical annotation on the touch-screen display.

10. The method according to claim 7, further comprising the step of:
following at least the dragging and dropping of the at least one graphical annotation tool to the anatomical image on the touch-screen display, displaying on the main display quantitative results of the at least one graphical annotation on the anatomical image.

11. The method according to claim 7, wherein
following at least the dragging and dropping of the at least one graphical annotation tool to the anatomical image on the touch-screen display, displaying on the touch-screen display a display mode displayed prior to the display of the at least one graphical annotation tool.

12. A touch-screen display for graphically annotating an anatomical image, the touch-screen display configured to interface with:
a main image display;
a processing unit; and
a network configured to interface the main image display with the processing unit and configured to interface the touch-screen display with the processing unit,
wherein the touch-screen display is configured such that an image displayed on the main display is concurrently displayed on the touch-screen display via the processing unit interfacing the main image display with the touch-screen display through the network, and
wherein the touch-screen display is configured such that the touch-screen display enables dragging and dropping at least one graphical annotation tool displayed on the main display from the main display to the anatomical image concurrently displayed on the touch-screen display by touching the at least one graphical annotation tool displayed on the touch-screen display.

13. The touch-screen display according to claim 12, wherein the touching of the at least one graphical annotation on the touch-screen display is effected by one of (a) a stylus; (b) a light pen; (c) a mouse; (d) a display screen thumbnail; (e) a track ball; (f) a joystick control; and (g) touching by a user.

14. The touch-screen display according to claim 12, wherein the touch-screen display is configured to enable at least one of positioning and varying of size of the at least one graphical annotation.

15. The touch-screen display according to claim 12, wherein
the touch-screen display is configured such that following graphical annotation of the anatomical image on the touch-screen display, the main display displays quantitative results of the at least one graphical annotation on the anatomical image.

16. The touch-screen display according to claim 12, wherein
the touch-screen display is configured such that following graphical annotation of the anatomical image on the touch-screen display, the touch-screen display displays a display mode displayed prior to the display of the at least one graphical annotation tool.

17. The touch-screen display according to claim 12, wherein the touch-screen display is positioned in proximity to a patient bed.
